(19) United States
(12) Patent Application Publication
(10) Pub. No.: US 2021/0128249 A1
(43) Pub. Date: May 6, 2021

(54) TRACKER DEVICE FOR COMPUTER-ASSISTED SURGERY

(71) Applicant: MEDTECH SA, Montpellier (FR)

(72) Inventors: Max BILLARD, Jacksonville, FL (US); Florian COISEUR, Montpellier (FR); Pierre MAILLET, Montpellier (FR)

(21) Appl. No.: 17/085,796

(22) Filed: Oct. 30, 2020

Related U.S. Application Data
(60) Provisional application No. 62/927,803, filed on Oct. 30, 2019.

(51) Int. Cl. A61B 34/20

(52) U.S. Cl. CPC A61B 34/20; A61B 2034/2074; A61B 2034/2055

(57) ABSTRACT

An assembly including a surgical instrument having a working end extending along an axis. A tracker device has an axisymmetric body or a quasi-axisymmetric body, the tracker device being mounted on the surgical instrument such that an axis of axisymmetry of the axisymmetric body or the quasi-axisymmetric body is collinear with the axis of the surgical instrument. A position of the surgical instrument is tracked as a function of a tracking of the axisymmetric body or quasi-axisymmetric body. A computer-assisted surgery system for tracking surgical instruments during surgery is provided.

### CROSS-REFERENCE TO RELATED APPLICATIONS

The present application claims the benefit of U.S. Provisional Patent Application No. 62/927,803, entitled, “TRACKER DEVICE FOR COMPUTER-ASSISTED SURGERY,” filed on Oct. 30, 2019, the contents of which are incorporated herein by reference.

### FIELD OF DISCLOSURE

The present disclosure relates to computer-assisted surgery systems and, more particularly, to instrumentation used for the tracking of surgical tools and patients during computer-assisted surgery.

### BACKGROUND

Tracking of surgical instruments or tools is an integral part of computer-assisted surgery (hereinafter CAS). The tools are tracked for position and/or orientation in such a way that information pertaining to bodily parts is obtained. The information is then used in various interventions with respect to the body, such as bone alterations, implant positioning, incisions and the like.

Optical tracking systems are commonly used in the operating room, notably because active transmitters on the tools are not required on the tools, and therefore represent fewer issues pertaining to sterilization. The CAS system associated with such passive tracking has an optical sensor apparatus provided to visually detect optical elements on the tools. The optical elements are passive, whereby no power source is associated therewith.

In order to obtain values for position and/or orientation, the optical elements must be in the line of sight of the optical sensor apparatus. Accordingly, with passive tracking systems, the surgeon or staff must maintain the required visibility between the optical sensor apparatus and the optical elements. This line of sight requirement may hamper movements of an operator or robot performing surgery.

### SUMMARY

Therefore, in accordance with an aspect of the present disclosure, there is provided an assembly including: a surgical instrument having a working end extending along an axis; and a tracker device having an axisymmetric body or a quasi-axisymmetric body, the tracker device configured to be mounted on the surgical instrument such that an axis of axisymmetry of the axisymmetric body or the quasi-axisymmetric body is collinear with the axis of the surgical instrument; whereby at least a position of the surgical instrument is tracked as a function of a tracking of the axisymmetric body or the quasi-axisymmetric body .

In accordance with another aspect of the present disclosure, there is provided a computer-assisted surgery system for tracking surgical instruments during surgery, including: a surgical instrument having a working end extending along an axis; and a tracker device including a body, the body having an axisymmetric body or a quasi-axisymmetric body, the tracker device configured to be mounted on the surgical instrument such that an axis of axisymmetry of the body is collinear with the axis of the surgical instrument; a tracking system having: a sensor unit configured to output a tracking output of the tracker device as the surgical instrument is moved; a database storing geometrical pattern data and instrument and tracker device relation data; and a position and orientation calculator configured to determine a position and an orientation of the tracker device as a function of the tracking output of the tracker device from the sensor unit. The tracking system is configured to calculate at least a position of the surgical instrument as a function of a tracking of the axisymmetric body or the quasi-axisymmetric body.

In accordance with yet another aspect of the present disclosure, a computer-assisted surgery system for tracking surgical instruments during surgery, including: a processing unit; and a non-transitory computer-readable memory communicatively coupled to the processing unit and including computer-readable program instructions executable by the processing unit for: obtaining image data of an axisymmetric body or a quasi-axisymmetric body of a tracker device, determining an orientation of the axisymmetric body or the quasi-axisymmetric body from the imaging, and calculating and outputting at least a position of a surgical instrument as a function of a tracking of the axisymmetric body or the quasi-axisymmetric body.

### BRIEF DESCRIPTION OF THE DRAWINGS

Having thus generally described the nature of one or more aspects of the present disclosure, reference will now be made to the accompanying drawings, showing by way of illustration a preferred embodiment thereof and in which:

FIGS. 1A, 1B and 1C are schematic views of tracker devices for computer-assisted surgery, in accordance with one or more aspects of the present disclosure; and

FIG. 2 is a block diagram of a computer-assisted surgery system using the tracker device of any one of FIGS. 1A, 1B and 1C.

### DETAILED DESCRIPTION

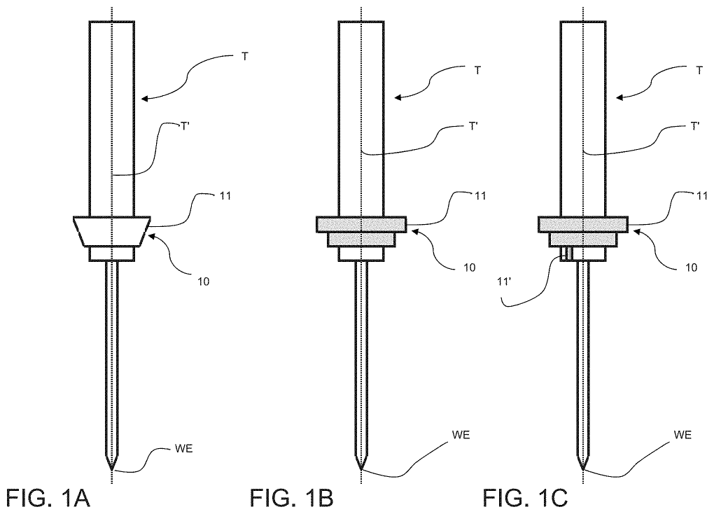

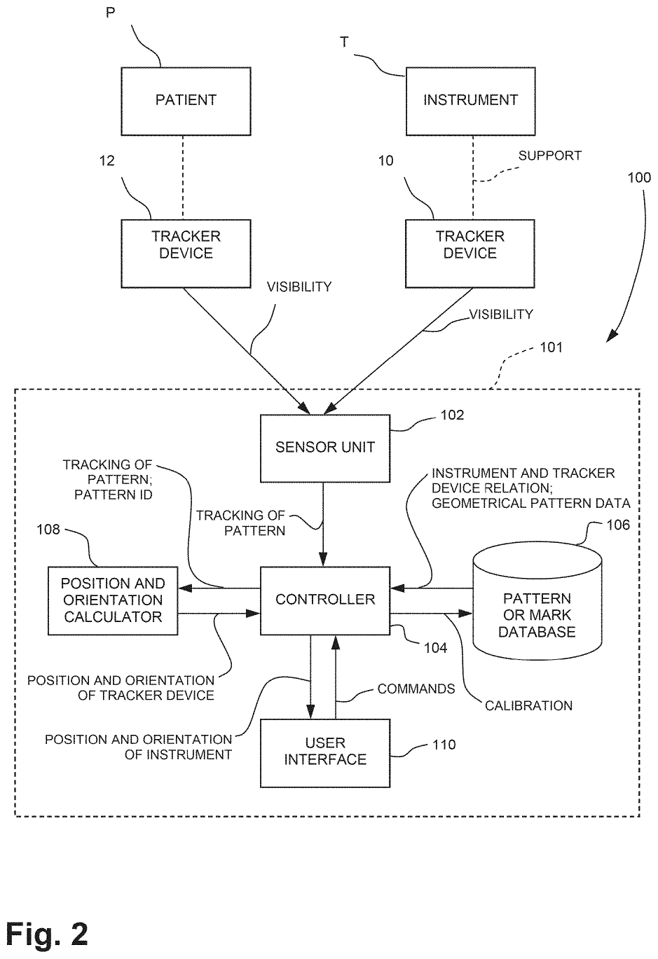

Referring now to the drawings and more particularly to FIGS. 1A, 1B and 1C, a tracker device in accordance with an embodiment is generally shown at 10, and is provided for the tracking of a surgical tool T. The surgical tool T may be of the type having a working end axis T′, which may be the longitudinal axis of the tool T, the axis T′ passing through the working tip WE and/or through the working end of the tool T. The axis T′ may be representative of a geometry of working action of the surgical tool T on a bone. For example, the surgical tool T may be a registration pointer, and awl, a rasp, a drill, a drill guide or tube, an impactor, elongated probe, needle, syringe or like instruments used in CAS. For simplicity, FIGS. 1A, 1B and 1C use a registration pointer as an example, but other tools T could be used as well. Moreover, the registration pointer of FIGS. 1A, 1B and 1C is shown as having a straight shaft at the end of which is the working tip WE, but could also have a section offset from the axis T′. The tracker device 10 is secured to the surgical tool T in a given geometrical relation, such that an optical sensor apparatus of a CAS system visually recognizes the tracker device 10 as detailed below. The tracker device 10 may be secured in any appropriate way, such as by a complementary mating engagement, with or without fasteners (e.g., set screw), through integral manufacturing, etc. With the tracking of the tracker device 10, the CAS system calculates a position and/or orientation of the surgical instrument or tool associated with the tracker device 10, including the longitudinal axis T′. One or more tracker device(s) 12 may also be used, as shown in FIG. 2. One such tracker device(s) 12 may be in a fixed relation relative to an anatomical feature of patient P, such as a bone, for instance by being screwed to the patient's bone or attached to it in an appropriate fashion. The tracker device(s) 12 is configured for being visually recognized by an optical sensor apparatus of a CAS system. For instance, the tracker device(s) 12 may have a pattern of three spheres, a geometrical shape, etc. for being visually recognized. In some aspects, the anatomical feature being tracked may not move (e.g., it may be strapped or retained), whereby the tracker device(s) 12 may not be necessary in such embodiments, or in other aspects in which the anatomical features are not tracked.

The tracker device 10, and possibly the tracker device 12, may have an axisymmetric body or an axisymmetric body portion, that may be viewed by a sensor apparatus via one or more peripheral surfaces 11. The axisymmetric body is mounted to the tool T in any appropriate way, such as to the stem, shaft, handle, etc. The axisymmetric body is secured to the tool T so as to have its axis of axisymmetry collinear with the axis T′ of the tool T. Accordingly, the axis of axisymmetry can be viewed as axis T′ in FIGS. 1A, 1B and 1C. Moreover, according to an aspect, the tracker device 10 may be shaped so as not to have any plane of symmetry to which the axis of axisymmetry would be normal. Stated differently, the axisymmetric body cannot be cut in symmetric halves by a cut transverse to the axis T′ in the page of the FIGS. 1A, 1B and 1C. However, in an alternate embodiment, the axisymmetric body has such a plane of symmetry.

In the embodiment of FIG. 1A, the axisymmetric body is shaped as a frusto-cone, also known as a truncated cone. The truncated cone may be oriented so as to taper toward the working end WE of the tool T. A central axis of the frusto-cone may be coincident with the axis T′. The reverse arrangement is possible as well, with the truncated cone flaring away from the working end WE of the tool T. The truncated cone is axisymmetric, in that its peripheral surface 11 continuously surrounds the axis of axisymmetry T′. The truncated cone is shown being on the handle, i.e., on the larger part of the tracker device 10, away from the working tip WE, but could be at other locations, such as on the shaft.

In the embodiment of FIG. 1B, the axisymmetric body is constituted of two discs, forming two distinct peripheral surfaces 11, with a step there between. The step may be present due to the discs having different diameters. In another embodiment, the discs are spaced apart from another, instead of being one against the other as in FIGS. 1B and 1C. There may be two or more discs, or a single disc with an irregular pattern, such as a wavy edge on one of two edges of the peripheral surface 11. Colour (color) schemes and/or contrasting patterns may also be used in similar fashion. For example, the frusto-cone of FIG. 1A and/or the discs in FIG. 1B, (e.g., one of the two discs, both discs) may have given colours, for being recognized as discussed below. The tracker device 10 of FIG. 1B may be oriented so as to have the small disc closer to the working end WE of the tool T. The reverse arrangement is possible as well. The tracker device 10 of FIG. 1B is axisymmetric, in that its peripheral surfaces 11 continuously surround the axis of axisymmetry T′. Moreover, the central axes of the discs may be coincident with the axis of axisymmetry T′ (i.e., they lie on one another). The tracker device 10 of FIG. 1B is shown as being on the handle, but could be at other locations, such as on the shaft.

In the embodiment of FIGS. 1A, 1B and 1C and in other embodiments, the axisymmetric body may be quasi-axisymmetric body, in that there may a disruption in the continuity of the peripheral surface(s) 11. Stated differently, the peripheral surface may not extend 360 degrees around the axis of axisymmetry T′, such as by having an axial slice removed from the frusto cone of FIG. 1A or discs or cylinders of FIGS. 1B and 1C. In an embodiment, the axisymmetric body is axisymmetric over at least 270 degrees around the axis of axisymmetry T′. The expression axis of axisymmetry T′ is used even though the axisymmetric body may be quasi-axisymmetric body.

Referring to FIG. 1C, the tracker device 10 has a configuration similar to that of the tracker device 10 of FIG. 1B, namely with an axisymmetric body constituted of two or more discs, forming two or more distinct peripheral surfaces 11, with a step there between. The discs may be spaced apart from another. In addition to the axisymmetric body, the tracker device 10 of FIG. 1C has a clocking feature 11′. The clocking features 11′ may also be known as an asymmetric feature relative to the axis of axisymmetry T′, and therefore relative to the axisymmetric body. In the illustrated embodiment, the clocking feature 11′ is a finger or like projection, that is separate from the discs of the tracker device 10. The finger may have any appropriate geometry, with outlines that may be rectangular, circular, polygonal, triangular, etc. Other configurations for the clocking feature 11′ may include one or more of the following: a non-reflective figure on the peripheral surfaces 11, a local shape or edge discontinuity in the peripheral surface(s) 11, an offset body relative to the peripheral surfaces 11 (e.g., a disc offset from the axis of axisymmetry T′), or a gap in a quasi-axisymmetric body. There may be more than one clocking feature 11′ for the tracker device 10, and multiple clocking features 11′ may be different from one another for proper indication of orientation. Moreover, while the clocking feature 11′ is shown on a tracker device 10 having a configuration similar to that of FIG. 1B, the clocking feature(s) 11′ may be on other axisymmetric trackers, such as the one of FIG. 1A.

The clocking feature(s) 11′ may be used for instance to enable a tracking of an orientation of the tool T. As an example, the tool T may have a blade as working end WE. The clocking feature 11′ may be indicative of where the sharp edge of the blade is. Therefore, the clocking feature(s) 11′ may be used when the working end WE of the tool is non axisymmetric, or has a non-axisymmetric feature, or when the working end WE has a given orientation. In an embodiment, if the working end WE of the tool T is purely axisymmetric, such as may be the case for some registration pointers as shown in FIGS. 1A to 1C, then the clocking feature(s) 11′ may be absent or may not be used in the tracking process.

The tracker device 10 may be visible to the sensor unit 102 of the tracking system 101 of FIG. 2. Therefore, in an embodiment, at least the peripheral surface(s) 11 and clocking feature(s) 11′ (if present) of the tracker device 10 is (are) made of a retro-reflective material that contrast from other materials in given lighting conditions, such as light emitted by the sensor unit of the tracking system. For instance, the material Scotch-Lite™ is suited to be used as retro-reflective surface.

Referring to FIG. 2, a computer-assisted surgery system using the tracker device 10 is generally illustrated at 100. The computer-assisted surgery system 100 may be of the type used by surgeons or like operators manipulating tools, or by robotized surgery systems. For instance, instrument T may be part of a robot or manipulated by a robot arm. The tracker device 10 and the tracker device(s) 12 may be a part of the computer-assisted surgery system 100, as secured to a surgical instrument T by way of a support or in any appropriate manner, such as those shown in FIGS. 1A, 1B and 1C, for example. The computer-assisted surgery system has a tracking system 101, which may be a computer having a processor. A non-transitory computer-readable memory may be communicatively coupled to the processing unit and including computer-readable program instructions executable, for instance in the form of a controller or a calculator module described below. The tracking system 101 has a sensor unit 102 provided in order to visually track the tracker device 10 and the tracker device(s) 12, if present, simultaneously with the tracking of one or more of the tracker device 10. Typically, the sensor unit 102 involves includes at least a pair of sensors (e.g., NDI Polaris). As other aspects, the sensor unit 102 may be a stereoscopic camera, a 3D camera, a motion detection camera, etc. A controller module 104 is connected to the sensor unit 102. Therefore, the controller module 104 receives a feed of images (i.e., image data, video) representative of the tracking of patterns from the sensor unit 102.

A database 106 may be provided so as to store the geometrical pattern data or other marks, such as optical patterns and/or colour schemes. More specifically, the various patterns and marks of the tracker device 10 and of the tracker device(s) 12, if present, are stored in the database 106. Similarly, the relation between the instrument and the tracker device 10 is stored in the database 106. According to an embodiment, the geometrical pattern data includes a 3D virtual model of the geometry of the axisymmetric body of the tracker device 10, including the axis of axisymmetry T′. The geometrical pattern data may include the clocking feature(s) 11′ relative to the 3D virtual model of the geometry of the axisymmetric body of the tracker device 10 that includes the axis of axisymmetry T′. The pattern and mark data may also include instrument/tracker device relation data. This relation data may be a position of the working end WE relative to the axisymmetric body, the position being along the axis of axisymmetry T′. This relation data may include an orientation of a portion of the working end WE (e.g., sharp edge) relative to the clocking feature(s) 11′. If the sensor unit 102 has the capacity of detecting colors, the colour schemes or like marks may be used to get additional information on the tracker(s) 10 and/or 12 and tools T. For example, a colour scheme or like mark may serve for the system 101 to identify a tool (e.g., probe is blue tracker, awl is red tracker) and/or to indicate the size of the tracker 10/12 (e.g., blue cone tracker is a 20.0 mm tracker, red cone tracker is a 15.0 mm tracker). The colour schemes or like marks may be used as clocking feature(s) 11′.

The instrument/tracker device relation may result from a calibration performed in the first steps of use of the computer-assisted surgery system. A calibration of the surgical tool T with the tracker device 10 thereon may be performed prior to the use of the tracker device 10, to calibrate a position of the working end WE of the tool T relative to the tracker device 10. The calibration of the surgical tool T with the tracker device 10 may be performed to calibrate an orientation of the working end WE of the tool T relative to a clocking feature(s) 11′ of the tracker device 10, if present or if desired.

A position and orientation calculator module 108 is associated with the controller module 104. The position and orientation calculator module 108 receives the tracking image feed of the axisymmetric body from the sensor unit 102, as well as the geometrical pattern data. The position and orientation calculator module 108 identifies which one of the patterns or marks of the tracker device 10 and of the tracker device(s) 12, if present, is being tracked, e.g., the axisymmetric body of FIG. 1A or FIG. 1B, or other, with or without a clocking feature(s) as in FIG. 1C. With the identification of the pattern or mark being tracked, the position and orientation calculator calculates the position and orientation of the tracker device 10 and the tracker device(s) 12, if present, as the perception of the axisymmetric body by the sensor unit 102 can be correlated to the dimensions from the database 106. Triangulation techniques may be used therefor, in that the two points of view from the sensor unit 102 enable the use of triangulation to determine the position and orientation of the tracker device 10 and tracker device(s) 12.

The position and orientation of the tracker device 10 is sent to the controller module 104. The controller module 104 will combine this information with the instrument/tracker device relation from the geometrical pattern database 106, so as to calculate or determine the position and orientation of the working end WE of the instrument T, along with axis of axisymmetry T′, for instance relative to the anatomical feature of the patient, such as a bone model, a digitized surface, etc. This may entail the tracking of the tracker device(s) 12 secured to the anatomical feature.

The geometry of the tracker device 10 and of the tracker device(s) 12, if present, is recognized by the tracking system 101 of the CAS system. The preferred absence of a plane of symmetry other than the one related to axisymmetry is such that the distance of the tracker device 10 and the tracker device(s) 12, if present, from the point of view of the sensor unit 102 can be determined, and with trigonometry using dimensions of the tracker device 10, the orientation may also be determined. Moreover, the axisymmetry and the collinear relation between the axis of axisymmetry T′ and axis of the surgical instrument T increase the range of visibility of the tracker device 10 and instrument to 360 degrees in some instances.

The user of the computer-assisted surgery system obtains information pertaining to the position and orientation of the instrument T via its axis of axisymmetry T′ and of the working end WE, in the various forms known to computer-assisted surgery (e.g., visual representation, numerical values such as angles, distances, etc.), via the user interface 110. It is pointed out that the database 106 may as well be part of the controller module 104 or the position and orientation calculator module 108, may be part of a cloud-based server, etc.

The system 100 may also be referred to as computer-assisted surgery system for tracking surgical instruments during surgery that has a processing unit; and a non-transitory computer-readable memory communicatively coupled to the processing unit and including computer-readable program instructions executable by the processing unit for: obtaining imaging of an axisymmetric body or quasi-axisymmetric body of a tracker device; determining an orientation of the axisymmetric body or quasi-axisymmetric body from the imaging; and calculating and outputting at least a position of a surgical instrument as a function of a tracking of the axisymmetric body or quasi-axisymmetric body, relative to an anatomical feature, such as fixed in a coordinate system or equipped with the tracker device(s) 12.

Therefore, the tracker devices 10 shown herein may be said to have, in some embodiment, a geometry of optical elements for use in CAS which are visible regardless of the devices' orientation relative to the sensor unit 102. Unlike a typical planar-based optical pattern, the cylindrical/axisymmetric pattern does not require the operator to actively maintain the tracker device's orientation with respect to the sensor unit 102.

In some embodiments, the tracker device 10 may be represented by an axisymmetric profile which the controller 104 uses to calculate the tracked objects central axis and position relative to other objects nearby and/or patient anatomy.

In some embodiments, the clocking feature(s) 11′ of the tracker device 10 may be represented by a partially axisymmetric profile, which provides the same information described above with additional features that indicate clocking of the axis to the tracking system 101 observing the tool.

For example, one of the discs of FIG. 1B may be eccentrically positioned on relative to the axis T′, essentially forming cams or lobes. Such a configuration, though related to axis T′ may not be axisymmetric.

In some embodiments, the tracker devices 10 have a lower profile than traditional planar optical elements. Stated differently, the volume or volumetric envelope of the tracker devices 10 may be less than standard planar-style optical devices (including support).

In some embodiments, the tracker device 10 could utilize colors/patterns to allow users to distinguish the tools/attachments more easily.

1. An assembly comprising:
a surgical instrument having a working end extending along an axis; and
a tracker device having an axisymmetric body or a quasi-axisymmetric body, the tracker device configured to be mounted on the surgical instrument such that an axis of axisymmetry of the axisymmetric body or the quasi-axisymmetric body is collinear with the axis of the surgical instrument;
whereby at least a position of the surgical instrument is tracked as a function of a tracking of the axisymmetric body or the quasi-axisymmetric body.

2. The assembly according to claim 1, wherein the axisymmetric body or the quasi-axisymmetric body is without any plane of symmetry to which the axis of axisymmetry is normal.

3. The assembly according to claim 1, wherein the axisymmetric body or the quasi-axisymmetric body includes a peripheral surface of retro-reflective material.

4. The assembly according to claim 1, wherein the tracker device includes the axisymmetric body, the axisymmetric body has a frusto-conical shape, with a central axis of the frusto-conical shape being collinear with the axis of axisymmetry.

5. The assembly according to claim 1, wherein the tracker device includes the axisymmetric body, the axisymmetric body has at least two discs, with central axes of the at least two discs being collinear with the axis of axisymmetry.

6. The assembly according to claim 1, wherein the tracker device includes the quasi-axisymmetric body, the quasi-axisymmetric body has a peripheral surface surrounding the axis of the surgical instrument by at least 270 degrees.

7. The assembly according to claim 1, wherein the tracker device further includes at least one clocking feature, the at least one clocking feature being non-axisymmetric, the at least one clocking feature being indicative of an orientation of the tracker device.

8. The assembly according to claim 7, wherein the working end of the surgical instrument has a non-axisymmetric feature.

9. The assembly according to claim 7, wherein the at least one clocking feature is a projection from the axisymmetric body or the quasi-axisymmetric body.

10. The assembly according to claim 1, wherein the axisymmetric body or the quasi-axisymmetric body has a mark indicative of an identity of the tracker device.

11. A computer-assisted surgery system for tracking surgical instruments during surgery, comprising:
a surgical instrument having a working end extending along an axis; and
a tracker device including a body, the body having an axisymmetric body or a quasi-axisymmetric body, the tracker device configured to be mounted on the surgical instrument such that an axis of axisymmetry of the body is collinear with the axis of the surgical instrument;
a tracking system having:
a sensor unit configured to output a tracking output of the tracker device as the surgical instrument is moved;
a database storing geometrical pattern data and instrument and tracker device relation data; and
a position and orientation calculator configured to determine a position and an orientation of the tracker device as a function of the tracking output of the tracker device from the sensor unit;
the tracking system configured to calculate at least a position of the surgical instrument as a function of a tracking of the axisymmetric body or the quasi-axisymmetric body.

12. The computer-assisted surgery system according to claim 11, wherein the axisymmetric body or the quasi-axisymmetric body is without any plane of symmetry to which the axis of axisymmetry is normal.

13. The computer-assisted surgery system according to claim 11, wherein the axisymmetric body or the quasi-axisymmetric body has a peripheral surface of retro-reflective material.

14. The computer-assisted surgery system according to claim 11, wherein the axisymmetric body has a frusto-conical shape, with a central axis of the frusto-conical shape being collinear with the axis of axisymmetry.

15. The computer-assisted surgery system according to claim 11, wherein the axisymmetric body has at least two discs, with central axes of the at least two discs being collinear with the axis of axisymmetry.

16. The computer-assisted surgery system according to claim 11, wherein the quasi-axisymmetric body has a peripheral surface surrounding the axis of the surgical instrument by at least 270 degrees.

17. The computer-assisted surgery system according to claim 11, wherein the tracker device further includes at least one clocking feature, the clocking feature being non-axisymmetric, the tracking system calculating an orientation a non-axisymmetric feature of the surgical instrument as a function of a tracking of the clocking feature.

18. The computer-assisted surgery system according to claim 17, wherein the at least one clocking feature includes a projection from the axisymmetric body or the quasi-axisymmetric body.

19. The computer-assisted surgery system according to claim 11, wherein the axisymmetric body or the quasi-axisymmetric body has a mark indicative of an identity of the tracker device, the tracking system identifying a surgical instrument using the mark.

20. A computer-assisted surgery system for tracking surgical instruments during surgery, comprising:
a processing unit; and
a non-transitory computer-readable memory communicatively coupled to the processing unit and comprising computer-readable program instructions executable by the processing unit for:
obtaining image data of an axisymmetric body or a quasi-axisymmetric body of a tracker device,
determining an orientation of the axisymmetric body or the quasi-axisymmetric body from the imaging, and
calculating and outputting at least a position of a surgical instrument as a function of a tracking of the axisymmetric body or the quasi-axisymmetric body.
